(19) United States
(12) Patent Application Publication
(10) Pub. No.: US 2014/0135591 A1
(43) Pub. Date: May 15, 2014

(54) FOOTWEAR CAPABLE OF BEING WIRELESSLY CHARGED AND TRANSMITTING BIOLOGICAL INFORMATION, METHOD FOR MANAGING HEALTH IN WIRELESS COMMUNICATION SYSTEM INCLUDING THE FOOTWEAR, AND WIRELESS COMMUNICATION SYSTEM TO WHICH THE METHOD IS APPLIED

(71) Applicant: Hanrim Postech Co., Ltd., Suwon-si (KR)

(72) Inventor: Chun-Kil JUNG, Seoul (KR)

(73) Assignee: Hanrim Postech Co., Ltd., Suwon-si (KR)

(21) Appl. No.: 14/074,484

(22) Filed: Nov. 7, 2013

(30) Foreign Application Priority Data
Nov. 9, 2012 (KR) 10-2012-0126930

(51) Int. Cl. A61B 5/00

(52) U.S. Cl. CPC A61B 5/6807; A61B 5/0015
USPC 600/301; 600/300

(57) ABSTRACT

Disclosed herein are a method for managing health in a wireless communication system including a footwear using a wireless charging technology, the method including obtaining a biological information of a user from a biological information detecting sensor of the footwear using a power obtained through a wireless power receiving device installed therein, and transmitting the biological information to an external electronic device via a communication module; a footwear able to be wirelessly charged and to transmit a biological information; and a wireless communication system which performs the method.

### CROSS-REFERENCE TO RELATED APPLICATIONS

This application claims the benefit of Korean Patent Application No. 10-2012-0126930, filed on Nov. 9, 2012, in the Korean Intellectual Property Office. All disclosures of the document named above are incorporated herein by reference.

### BACKGROUND OF THE INVENTION

1. Field of the Invention

The present invention relates to a footwear able to be wirelessly charged and to transmit biological information; a method for managing health in a wireless communication system including the footwear; and a wireless communication system which performs the method.

2. Description of the Related Art

In accordance with the development of a medical technology, it has become desirable to monitor a biological signal of a patient remotely. For example, an electrocardiogram monitoring system for diagnosing the state and/or disease of a heart may guide an electrocardiogram waveform for a short period of time. However, in order to observe a heart problem that is intermittently generated, such as an arrhythmia, it is necessary to continuously record and analyze the heart problem for a long period of time.

In addition, any means capable of coping with a rapid risk generated due to sleep apnea or the like, while a patient is in a location remote from medical facilities such as the home, have not been suggested.

According to the related art, it is impossible to measure a biological signal of a patient in real time, and an action may not be immediately taken when the patient is in an emergency state. In order to solve these problems, various studies and developments have been regularly conducted.

Korean Patent Application No. 10-1995-0029913 (entitled “System of Monitoring Bio Signal in Real Time by Using Wireless Communication Network”; hereinafter, referred to as the “related patent technology”) discloses a technology capable of diagnosing and preventing a disease of a patient by continuously checking a biological signal of the patient via a biological signal measuring device, the system transmitting a signal indicating an abnormality in the biological signal to a biological signal monitoring server device of a hospital in real time via a wireless communication network, and transmitting a diagnosing and emergency treating method from a biological signal monitoring center of the hospital to the biological signal measuring device of the patient in real time.

### SUMMARY OF THE INVENTION

An object of the present invention is to provide a method for managing health in a wireless communication system including a footwear using a wireless charging technology, the method obtaining biological information collected by the footwear after being worn by a user for a long time period, and coping with emergency situations using the biological information; and a wireless communication system which performs the method.

According to an embodiment of the present invention, there is provided a method for managing health using a wireless communication system including a footwear to which a wireless charging technology is applied, the method including: obtaining a biological information of a user from a biological information detecting sensor of the footwear using a power obtained through a wireless power receiving device installed therein; and transmitting the biological information to an external terminal via a communication module.

The method may further include storing an ID information in a memory installed in the footwear, wherein the transmitting of the biological information to the external terminal via the communication module includes: transmitting the ID information to the external terminal via the communication module; and transmitting the biological information to the external terminal via the communication module when an ID confirmation signal is received from the external terminal.

The external terminal may be a mobile communication terminal having a terminal ID information stored therein, and the method may further include transmitting the terminal ID information and the biological information to a health management server via a mobile communication module of the mobile communication terminal.

The biological information may include one or more of a pulse information, a blood pressure information, a body temperature information, an exercising amount information, and a time information.

The method may further include: receiving an alarm information generated based on the biological information via the communication module of the footwear; and outputting an alarm sound to a sound output module installed in the footwear, based on the alarm information.

According to another embodiment of the present invention, there is provided a method for managing health using a wireless communication system including a footwear to which a wireless charging technology is applied, the method including: receiving a biological information and an ID information of a user from a biological information detecting sensor of the footwear using a power obtained via a wireless power receiving device installed therein; analyzing the biological information to generate an alarm information; and transmitting the alarm information to an external terminal.

The analyzing of the biological information to generate the alarm information may include: storing a personal database in a memory; obtaining a health record information of the user in the personal database by using the ID information of the user; and generating the alarm information by using the health record information and the biological information.

According to still another embodiment of the present invention, there is provided a footwear able to be wirelessly charged and to transmit a biological information, the footwear including: an insole; an outer cover installed on the insole and having a wear space formed therein; a heel attached to a lower surface of the insole; a secondary coil installed in any one of the insole, the outer cover, and the heel and receiving a wireless power signal from an external wireless charging device; a battery installed in any one of the insole, the outer cover, and the heel and charged with an induced electromotive force generated in the secondary coil; a biological information detecting sensor installed in the wear space; a communication module installed at any one of the insole, the outer cover, and the heel; and a controller configured to charge the battery by using the wireless power signal received through the secondary coil and to transmit the biological information obtained from the biological information detecting sensor to an external electronic device via the communication module.

The footwear may further include a memory configured to store an ID information of a user, wherein the controller is configured to transmit the ID information to the external electronic device via the communication module, and to transmit the biological information to the external electronic device via the communication module when an ID confirmation signal is received from the external electronic device via the communication module.

The biological information may include one or more of a pulse information, a blood pressure information, a body temperature information, an exercising amount information, and a time information.

The footwear may further include a sound output module installed in the footwear, and the controller may be configured to output an alarm sound to a sound output module based on an alarm information generated based on the biological information when the alarm information is received via the communication module of the footwear.

The footwear may further include a position information module, wherein the controller is configured to transmit a position information obtained from the position information module to the external electronic device via the communication module when the alarm information is received via the communication module.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a diagram of a wireless communication system associated with a method for managing health according to an embodiment of the present invention;

FIG. 2 is a side view of a footwear able to be wirelessly charged and to transmit a biological information according to an embodiment of the present invention;

FIG. 3 is a cross-sectional view of the footwear shown in FIG. 2;

FIG. 4 is a block diagram of electronic components of the footwear able to be wirelessly charged and to transmit a biological information according to an embodiment of the present invention; and

FIG. 5 is a flow chart describing the method for managing health in a wireless communication system according to an embodiment of the present invention.

### DETAILED DESCRIPTION OF THE EMBODIMENTS

Reference will now be made in detail to the present embodiments of the present invention, examples of which are illustrated in the accompanying drawings, wherein like reference numerals refer to the like elements throughout. The embodiments are described below in order to explain the present invention by referring to the figures.

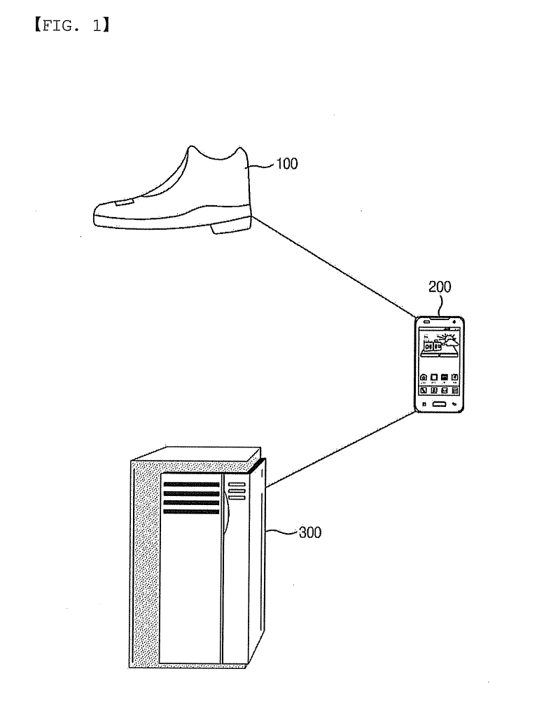

FIG. 1 is a diagram describing a wireless communication system associated with a method for managing health according to an embodiment of the present invention. As shown in FIG. 1, the wireless communication system is configured to include a footwear 100, a mobile communication terminal 200, and a health management server 300.

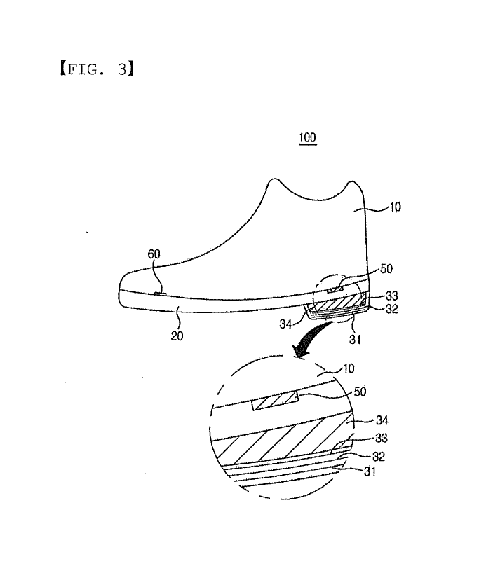

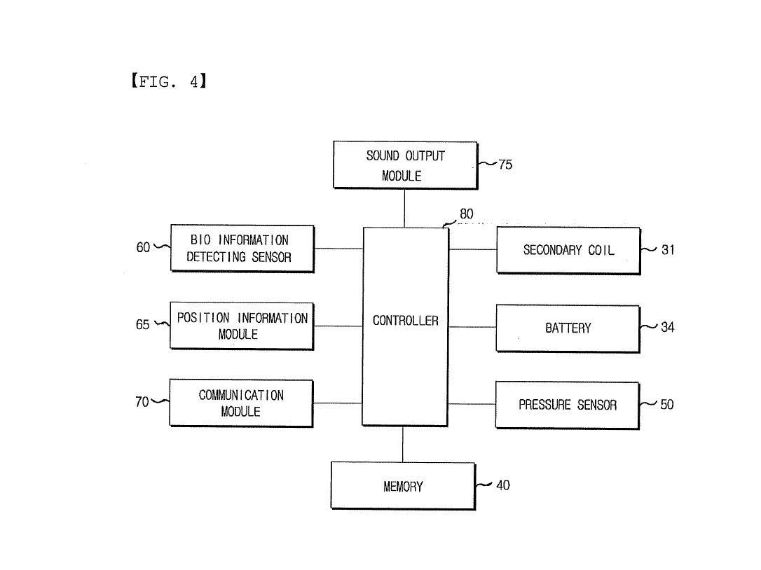

The footwear 100 transmits a biological information obtained through a biological information detecting sensor 60 (see FIGS. 3 and 4) installed therein to the mobile communication terminal 200, and the mobile communication terminal 200 obtaining the biological information transmits the biological information to the health management server 300 via a mobile communication module installed therein. The mobile communication terminal 200 and the footwear 100 may communicate with each other via a near field communication module using Bluetooth, Zigbee, or other similar protocols.

Meanwhile, the health management server 300 obtaining the biological information confirms a health state of a user by using the biological information, and periodically transmits a current health state to the mobile communication terminal. In the case of an emergency situation—that is, in the case in which a blood pressure rapidly becomes high or low or a body temperature rapidly becomes higher than a set temperature—the health management server 300 transmits an alarm information to the mobile communication terminal 200.

Although an example in which the biological information is transmitted to the health management server 300 via the mobile communication terminal 200 has been described in the above embodiment of the present invention, the present invention is not limited thereto. For example, the biological information may be directly transmitted from the footwear 100 to the health management server 300. That is, the footwear may include a mobile communication module rather than a near field communication module, and the biological information may be directly transmitted, together with an ID information stored in a memory of the footwear 100, to the health management server 300.

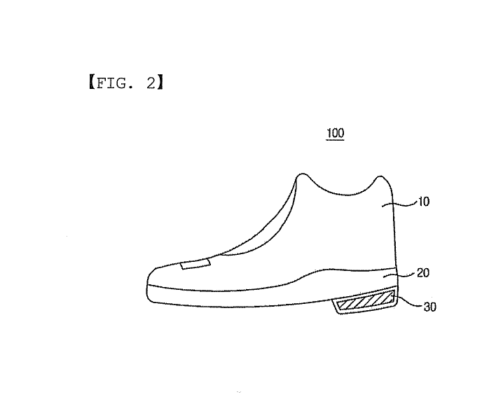

Hereinafter, a footwear 100 able to be wirelessly charged and to transmit a biological information according to an embodiment of the present invention will be described with reference to FIGS. 2 to 4.

FIG. 2 is a side view of a footwear able to be wirelessly charged and to transmit a biological information according to an embodiment of the present invention, and FIG. 3 is a cross-sectional view of the footwear shown in FIG. 2. As shown in FIGS. 2 and 3, the footwear 100 includes an insole 20, an outer cover 10 formed on the insole 20 and having a wear space formed therein, and a heel 30 attached to a rear part of the insole 20.

As shown in FIG. 3, a biological information detecting sensor 60 which can obtain a biological information of a user is attached to the insole 20. The heel 30 may comprise a wireless power receiving device, the wireless power receiving device comprising a secondary coil 31 configured to receive a wireless power signal and a rectifying module (not shown) configured to rectify an induced electromotive force generated from the secondary coil 31.

The heel 30 may further comprise a battery 34 which can be charged with power from the secondary coil 31, a board 33 on which is installed a communication module 70 (see FIG. 4) which transmits a biological information obtained by the biological information detecting sensor 60 to the mobile communication terminal 200, and a shielding plate 32 installed on the secondary coil 31 which prevents a leaked magnetic field from affecting the board 33 or the battery 34. A controller 80 (see FIG. 4) configured to control the wireless power receiving device, the battery 34, and the biological information detecting sensor 60 may also be installed on the board 33 or elsewhere.

Here, the secondary coil 31 may include a first sub coil, and a second sub coil installed on the first sub coil so as to overlap or be overlapped with the first sub coil, in order to increase a transmission efficiency. That is, in a case where a cross-sectional width of the heel is narrow, such that it is difficult to arrange the secondary coil 31 such that it has a sufficient size, two coils may be installed to overlap each other, thereby making it possible to increase a reception efficiency of the wireless power signal.

In addition, a pressure sensor 50 may be attached to a portion corresponding to the heel to control an activation and deactivation of the biological information detecting sensor 60. That is, through the pressure sensor 50, an operation of the biological information detecting sensor 60 may start when it is judged that the user is wearing the footwear 100 (e.g., when a pressure is detected) and stop when it is judged that the user is not wearing the footwear 100 (e.g., when a pressure is not detected). Alternatively, the controller 80 may operate the biological information detecting sensor 60 for a predetermined period based on a signal from the pressure sensor 50.

Meanwhile, a flexible circuit board (not depicted) may be used in order to connect the board 33 and the biological information detecting sensor 60 to each other. When a person walks or runs, the insole 20 of the footwear 100 is bent. In this case, when the flexible circuit board is used as an electrical connection means between the biological information detecting sensor 60 and the board 33, even though the insole 20 is bent, durability is not affected at all.

FIG. 4 is a block diagram describing an electronic configuration of the footwear according to an embodiment of the present invention. As shown in FIG. 4, the footwear 100 may be configured to include the secondary coil 31, the battery 34, a memory 40, the pressure sensor 50, the biological information detecting sensor 60, a position information module 65, a communication module 70, a sound output module 75, and the controller 80. For simplification reasons, previous paragraphs already describing operations of these components will not be duplicated here.

As shown in FIG. 4, according to an embodiment of the present invention, an ID information of a user is stored in the memory 40. The ID information of the user may be generated at the time of purchasing the footwear, or at a later time using the mobile communication terminal 200. The ID information of the user is transmitted together with the biological information to the mobile communication terminal 200 via the communication module 70. Alternatively, the ID information of the user is first transmitted by the footwear 100, the mobile communication terminal 200 transmits an ID confirmation signal to the footwear 100 if an ID information of the terminal and the ID information of the user are matched to each other, and the footwear 100 transmits the biological information to the mobile communication terminal 200 if it receives the ID confirmation signal.

Meanwhile, the footwear 100 further includes the sound output module 75 and the position information module 65. After the biological information is transmitted to the health management server 300 (previously described in FIG. 1) and the health management server 300 performs an analysis using the biological information, if it is judged that the user is in an emergency situation, an alarm information is generated and is received by the footwear 100. In this case, the controller 80 may operate the sound output module 75 to output an alarm sound, thereby asking people around the user for help. In addition, the controller 80 may transmit an emergency rescue request signal together with a position information obtained through the position information module 65 to an emergency rescue system (not depicted) via the mobile communication terminal 200. The emergency rescue system receiving the emergency rescue request signal may dispatch an emergency rescue team by using the position information.

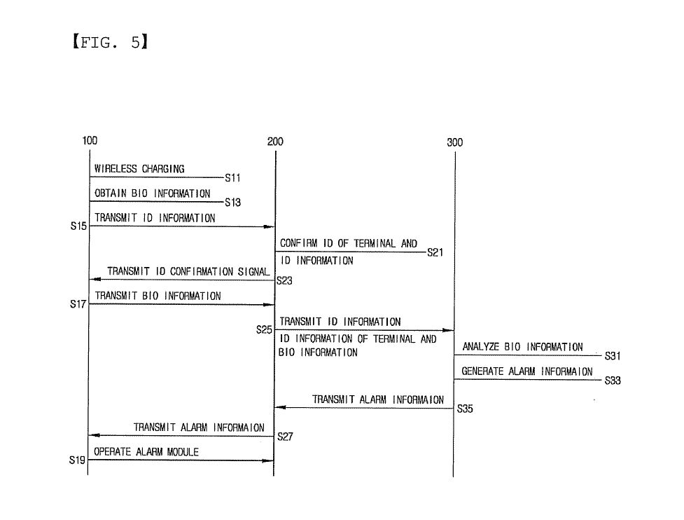

Hereinafter, a method for managing health in a wireless communication system including the footwear 100 having the above-described configuration will be described in detail with reference to FIG. 5.

FIG. 5 is a flow chart describing a method for managing health in a wireless communication system according to an embodiment of the present invention. As shown in FIG. 5, first, the biological information of the user is obtained from the biological information detecting sensor 60 of the footwear 100 by using wireless power obtained through the wireless power receiving device installed therein (S11 and S13). Meanwhile, the ID information of the user is stored in the memory 40 installed in the footwear 100. The ID information of the user stored in the memory 40 is transmitted to the mobile communication terminal 200 via the communication module 70 (S15). The mobile communication terminal 200 confirms whether the ID information of the user and the ID information of the terminal are matched to each other (S21), and if so transmits an ID confirmation signal to the footwear 100 (S23). Then, the biological information is transmitted to the external terminal 200 (mobile communication terminal) via the communication module 70 (S17). The mobile communication terminal 200 obtaining the biological information transmits the ID information of the terminal (or the ID information of the user) and the biological information to the health management server 300 via the mobile communication module installed therein (S25).

The biological information may include one or more of a pulse information, a blood pressure information, a body temperature information, an exercising amount information, and a time information, among other types of information.

A personal database is stored in a memory of the health management server 300, and the health management server 300 obtains a health record information of the user from the personal database by using the ID information and performs a health analysis by using the health record information and the biological information (S31). When it is judged from the health analysis that the user is in an emergency situation, the health management server 300 generates the alarm information (S33). The generated alarm information is transmitted to the footwear 100 via the mobile communication terminal 200 (S35 and S27).

The footwear 100 receiving the alarm information operates an alarm module 75 (sound output module) installed therein to output an audio for asking persons around the user for help and operates the position information module 65 to obtain the position information and transmit the obtained position information to the emergency rescue system (operations not depicted), thereby requesting the emergency rescue team.

The health management server 300 may also periodically transmit a health state information, based on the health analysis, to the mobile communication terminal 200 even when there is not an emergency situation (operation not depicted).

According to an embodiment of the present invention having the above-mentioned configuration, since the battery is charged using a wireless charging technology, a completely waterproof footwear may be manufactured at a cheap cost, and a difficulty in replacing or accessing the battery may be avoided.

In addition, according to an embodiment of the present invention, since biological information may be obtained from the footwear for a long period of time, basic data through which a health state of the user may be more accurately judged may be obtained.

Further, according to an embodiment of the present invention, when it is judged that the health state of the user is an emergency state, the user may be rescued by using the position information of the footwear.

Although a few embodiments of the present invention have been shown and described, it would be appreciated by those skilled in the art that changes may be made in this embodiment without departing from the principles and spirit of the invention, the scope of which is defined in the claims and their equivalents. In particular, all or some of the above mentioned embodiments may be selectively combined with each other so that they may be variously modified.

What is claimed is:

1. A method for managing health using a wireless communication system including a footwear, the method comprising:
obtaining a biological information of a user from a biological information detecting sensor of the footwear; and
transmitting the biological information to an external terminal via a communication module.

2. The method of claim 1, further comprising storing an ID information in a memory installed in the footwear,
wherein the transmitting of the biological information to the external terminal via the communication module comprises:
transmitting the ID information to the external terminal via the communication module; and
transmitting the biological information to the external terminal via the communication module when an ID confirmation signal is received from the external terminal.

3. The method of claim 2, wherein the external terminal is a mobile communication terminal having a terminal ID information stored therein, and
the method further comprises transmitting the terminal ID information and the biological information to a health management server via a mobile communication module of the mobile communication terminal.

4. The method of claim 1, wherein the biological information comprises one or more of a pulse information, a blood pressure information, a body temperature information, an exercising amount information, and a time information.

5. The method of claim 1, further comprising:
receiving an alarm information generated based on the biological information via the communication module of the footwear; and
outputting an alarm sound to a sound output module installed in the footwear, based on the alarm information.

6. A method for managing health in a wireless communication system including a footwear, the method comprising:
receiving a biological information and an ID information of a user from a biological information detecting sensor of the footwear;
analyzing the biological information to generate an alarm information; and
transmitting the alarm information to an external terminal.

7. The method of claim 6, wherein the analyzing of the biological information to generate the alarm information comprises:
storing a personal database in a memory;
obtaining a health record information of the user in the personal database by using the ID information of the user; and
generating the alarm information by using the health record information and the biological information.

8. A footwear comprising:
an insole;
an outer cover installed on the insole and having a wear space formed therein;
a heel attached to a lower surface of the insole;
a secondary coil installed in any one of the insole, the outer cover, and the heel, and receiving a wireless power signal from an external wireless charging device;
a battery installed in any one of the insole, the outer cover, and the heel, and charged with an induced electromotive force generated in the secondary coil;
a biological information detecting sensor installed in the wear space;
a communication module installed at any one of the insole, the outer cover, and the heel; and
a controller configured to transmit the biological information obtained from the biological information detecting sensor to an external electronic device via the communication module.

9. The footwear of claim 8, further comprising a memory configured to store an ID information of a user,
wherein the controller is configured to transmit the ID information to the external electronic device via the communication module, and to transmit the biological information to the external electronic device via the communication module when an ID confirmation signal is received from the external electronic device via the communication module.

10. The footwear of claim 8, wherein the biological information comprises one or more of a pulse information, a blood pressure information, a body temperature information, a exercising amount information, and a time information.

11. The footwear of claim 8, further comprising a sound output module installed in the footwear,
wherein the controller is configured to output an alarm sound to the sound output module based on an alarm information generated based on the biological information when the alarm information is received via the communication module of the footwear.

12. The footwear of claim 11, further comprising a position information module,
wherein the controller is configured to transmit a position information obtained from the position information module to the external electronic device via the communication module when the alarm information is received via the communication module.

13. The method of claim 1, wherein the biological information detecting sensor of the footwear uses a power obtained through a wireless power receiving device installed in the footwear.

14. The method of claim 6, wherein the biological information detecting sensor of the footwear uses a power obtained through a wireless power receiving device installed in the footwear.

15. The method of claim 5, further comprising, after receiving the alarm information, transmitting a position information obtained from a position information module to the external electronic device via the communication module.

16. The method of claim 6, further comprising:
after transmitting the alarm information, receiving a position information of the footwear; and
transmitting the position information to an emergency rescue system.

17. The footwear of claim 8, wherein the controller is further configured to charge the battery using the wireless power signal received through the secondary coil.

18. A wireless communication system comprising:
the footwear of claim 8;
a mobile communication terminal in communication with the communication module of the footwear; and
a health management server in communication with the mobile communication terminal,
wherein the mobile communication terminal serves as the external electronic device.

19. A wireless communication system comprising:
the footwear of claim 8; and
a health management server in communication with the communication module of the footwear,
wherein the health management server serves as the external electronic device.
